(19) United States
(12) Patent Application Publication
(10) Pub. No.: US 2008/0183083 A1
(43) Pub. Date: Jul. 31, 2008

(54) SYSTEMS AND METHODS FOR MONITORING EFFECTIVENESS OF CONGESTIVE HEART FAILURE THERAPY

(76) Inventors: H. Toby Markowitz, Roseville, MN (US); Sameh Sowelam, Fridley, MN (US)

Correspondence Address: MEDTRONIC, INC., 710 MEDTRONIC PARKWAY NE, MINNEAPOLIS, MN, 55432-9924, US

(21) Appl. No.: 11/669,396

(22) Filed: Jan. 31, 2007

(51) Int. Cl. A61B 5/0205; A61N 1/36

(52) U.S. Cl. 600/484; 607/9

(57) ABSTRACT

A method for monitoring a patient includes measuring a series of consecutive pulse transit times (PTT's) of the patient, and processing the resulting PTT signal to detect a presence or absence of central sleep apnea (CSA). The method further includes determining an effectiveness of congestive heart failure therapy, which is being provided to the patient, based on the detected presence or absence of CSA. A system incorporating the method includes an electrode of an implantable medical device, which is adapted to pick up the patient's ventricular depolarization signals, a sensor, which is adapted to pick up peripheral arterial pulse signals of the patient, and a signal processor, which is adapted to receive the two types of signals and to process the signals according to the method. The system may provide the therapy via cardiac resynchronization pacing and, upon detection of CSA, the system may adjust at least one pacing parameter.

### TECHNICAL FIELD

The present invention pertains to congestive heart failure (CHF) therapy and more particularly to sleep apnea monitoring and classification, utilizing an implanted medical device, to evaluate an effectiveness of CHF therapy delivered from the device.

### BACKGROUND

Because congestive heart failure (CHF) may cause and/or be caused by a person's abnormal breathing patterns, including periodic breathing, particularly manifest in the form of sleep apnea, sleep apnea may be an indication of developing heart failure in that person. In general, there are two types of sleep apnea, obstructive and central. Obstructive sleep apnea (OSA), which is caused by an airway obstruction, for example, collapse of the pharynx, can adversely impact attempts to treat heart failure. Central sleep apnea (CSA) is frequently associated with CHF, and may be a manifestation of worsening CHF. Because of the limited response of the heart suffering from CHF to supply blood, to meet demand, blood CO₂ levels, which are detected by peripheral vascular chemoreceptors, change slowly. This slow response may introduce control system instability in the physiological loop that regulates breathing; this instability leads to periodic breathing in which respiration fluctuates between hypopnea/apnea and hyperpnea. A well known type of periodic breathing is known as Cheyne-Stokes Respiration (CSR).

In recent years implantable medical devices (IMD's) have been adapted to treat congestive heart failure via bi-ventricular pacing, which provides cardiac resynchronization therapy (CRT). Further adaptation of these types of devices, for the detection and therapeutic treatment of sleep apneas, has been described, for example, in commonly-assigned patent application Ser. No. 10/419,404, entitled APPARTAUS AND METHOD FOR MONITORING FOR DISORDERED BREATHING, salient portions of which are hereby incorporated by reference. The effectiveness of congestive heart failure therapy is typically monitored via measurement of one or more hemodynamic parameters, examples of which include, intra-cardiac pressure and left ventricular ejection fraction. The detection of sleep apnea events can provide another means for monitoring the effectiveness of heart failure therapy. However, because not all types of sleep apnea are influenced by heart failure, there is a need for monitoring systems and methods that can distinguish between the types of sleep apnea.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic depiction of various elements that may be incorporated by a system, according to some embodiments of the present invention.

FIG. 2 is an exemplary functional block diagram for an implantable medical device such as is shown in FIG. 1, according to some embodiments of the present invention.

FIG. 3 is a group of tracings illustrating a measure of pulse transit time, according to some embodiments of the present invention.

FIG. 4A is a plot representative of a pulse transit time signal corresponding to a central sleep apnea event.

FIG. 4B is a plot representative of a pulse transit time signal corresponding to an obstructive sleep apnea event.

FIG. 5 is a flow chart defining some methods of the present invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions, and manufacturing processes are provided for selected elements, and all other elements employ that which is known to those of skill in the field of the invention. Those skilled in the art will recognize that many of the examples provided have suitable alternatives that can be utilized.

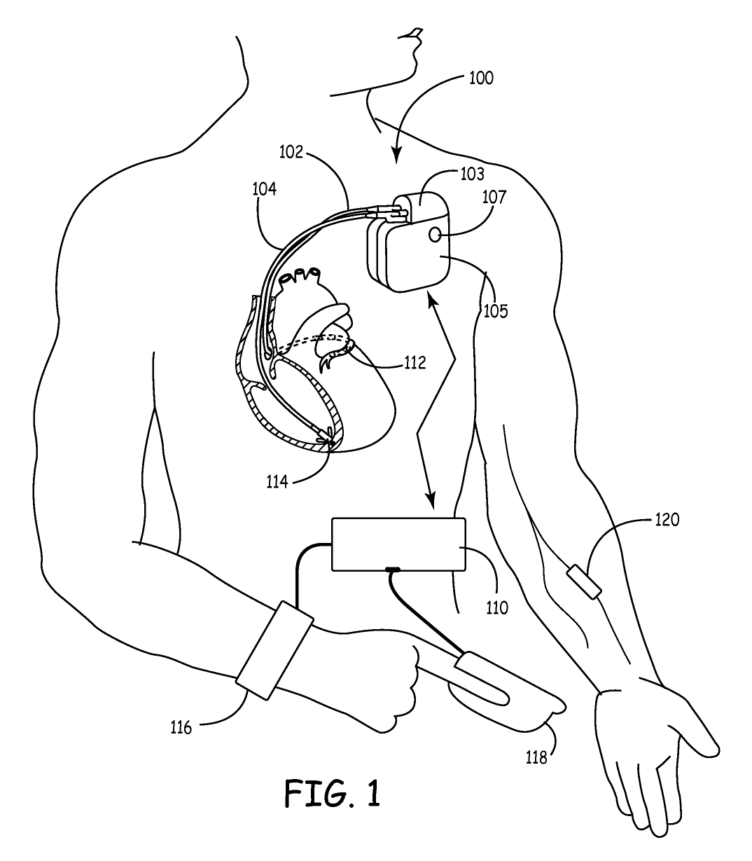

FIG. 1 is a schematic depiction of various elements that may be incorporated by a system, according to some embodiments of the present invention. FIG. 1 illustrates an IMD 100 implanted in a patient and including a first electrical lead 102, a second electrical lead 104, and a device housing 105 on which a connector module 103 is mounted to facilitate the coupling of leads 102, 104 to a battery and electronic components (not shown) enclosed within housing 105; configurations and construction details concerning such housing and connector module couplings for electrical leads are well known to those skilled in the art. First lead 102 is shown implanted within a coronary vein and including an electrode 112 positioned for sensing and stimulation of a left ventricle (LV) of the patient's heart, while second lead 104 is shown implanted in a right ventricle (RV) and including a tip electrode 114 positioned in an apex of the RV for sensing and stimulation in conjunction with that of LV electrode 112. Although not shown, IMD 100 may further include another electrode positioned in a right atrium (RA) of the patient's heart, either coupled to one of leads 102, 104 or coupled to another, atrial lead (not shown). According to the illustrated embodiment, IMD 100 is adapted to provide CRT via bi-ventricular pacing carried out by at least, LV electrode 112 and RV electrode 114, according to methods known to those skilled in the art.

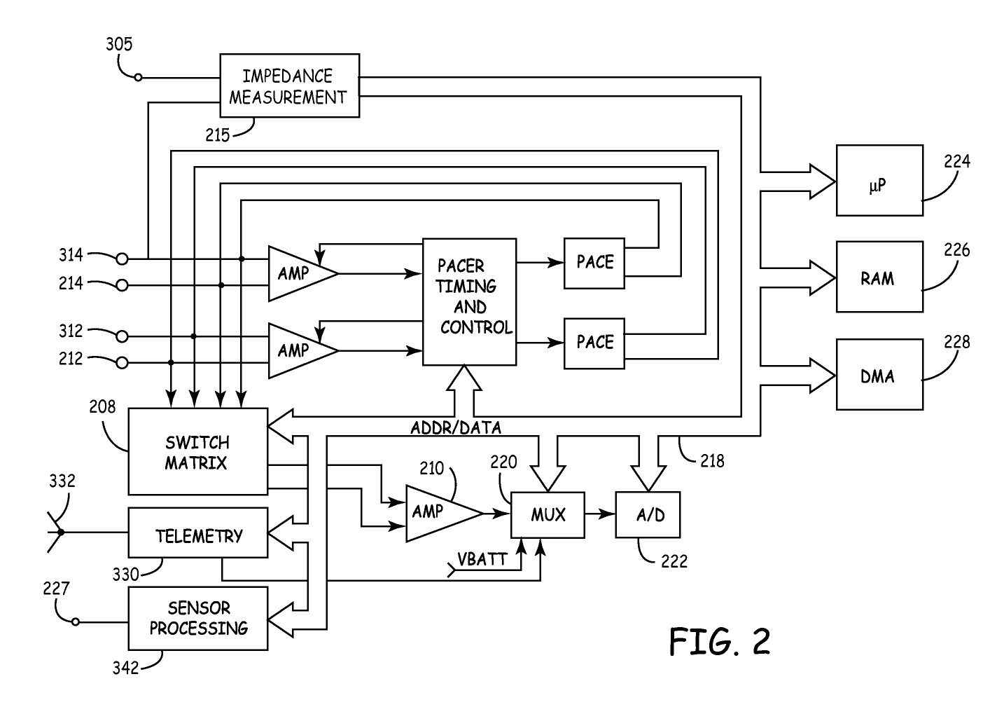

FIG. 2 is an exemplary functional block diagram for the electronic components enclosed within housing 105 of IMD 100, according to some embodiments of the present invention. Each of the aforementioned electrodes 112, 114 of leads 102, 104 is electrically coupled, via a conductor extending within leads 102, 104, to a connector of each lead 102, 104, each of which are electrically coupled to an electrical contact within connector module 103; the contacts within module 103 are coupled via electrical feedthroughs to terminals 212 and 214, which correspond to electrodes 112 and 114 respectively. Each of electrodes 112, 114 may be one of a bipolar pair, for example, FIG. 2 shows a terminal 314 which may correspond to another electrode forming a bipolar pair with electrode 114, and a terminal 312 which may correspond to another electrode forming a bipolar pair with electrode 112. According to the illustrated embodiment, terminals 212, 312, 214 and 314 electrically connect corresponding electrodes to sense amplifiers which provide the appropriate signals to a pacer timing and control circuit 212 according to respective preset thresholds. FIG. 2 further illustrates a switch matrix 208, under control of a microprocessor/controller 224, which is used to select, via bus 218, the electrodes which are to be coupled to a wide band amplifier 210 for use in digital signal analysis; the signals from the selected electrodes are directed through a multiplexer 220 and thereafter converted by an A/D converter 222 for storage in random access memory (RAM) 226, which is under the control of a direct memory access (DMA) circuit 228. Microprocessor 224 includes an associated ROM for storing programs that allow microprocessor 224 to analyze signals, transmitted thereto via bus 218, and to control the delivery of the appropriate therapy, for example, via pacing timing and control circuitry 212.

FIG. 1 further illustrates an external signal processor 110 hardwired to an external pressure cuff sensor 116, for example of the type used for blood pressure monitoring, and to a pulse-oximeter sensor 118, for example, a PureLight® sensor commercially available from Nonin Medical, Inc. of Plymouth, Minn. An implantable pressure cuff sensor 120, for example, as is described in commonly assigned U.S. Pat. No. 6,106,477, salient portions of which are hereby incorporated by reference, is also shown coupled to a radial artery, and an implantable pulse-oximeter sensor 107 is shown mounted to IMD housing 105. FIG. 2 further illustrates a terminal 227 for electrically connecting either of sensors 107, 120 to sensor processing circuitry 342, which is coupled to microprocessor 224 via data/address bus 218, for the transmission of sensor signals.

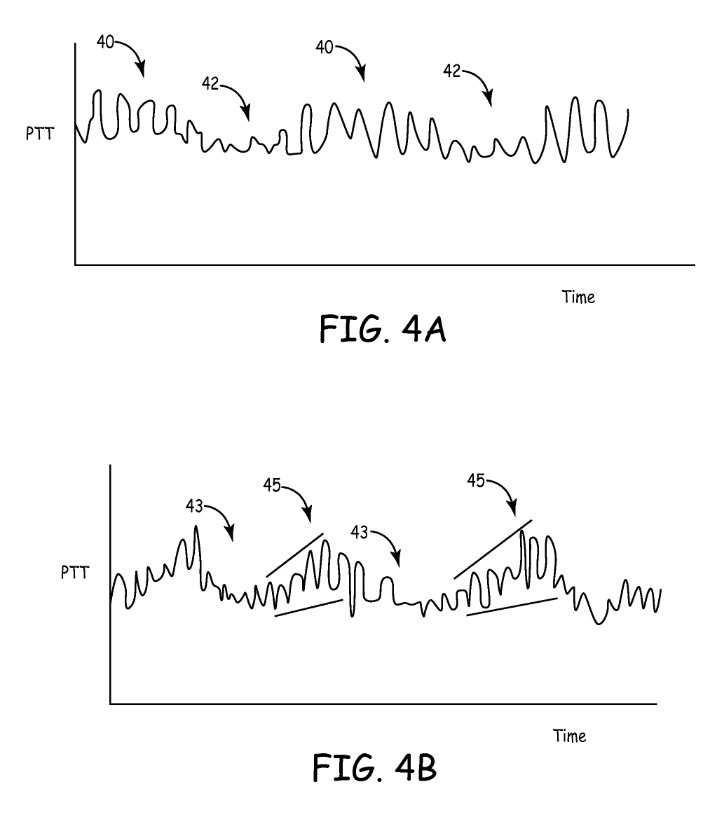

According to embodiments of the present invention, a system for monitoring an effectiveness of CRT delivered by IMD 100, via leads 102, 104, employs a monitoring method in which times for blood pulses to travel between two arterial sites are measured, collected and analyzed, either by signal processor 224 of IMD 100, or by external processor 110; the system includes electrode 114 to detect ventricular depolarization, and any one of sensors 107, 116, 118 and 120 to pick up a pulse signal downstream of the patient's heart. The time that it takes an arterial pulse to travel from the left ventricle, at aortic valve opening, to a arterial peripheral site, downstream, is known as a pulse transit time (PTT); PTT is typically measured as the time delay between each detected ventricular depolarization and each subsequent peripheral pulse signal. PTT signals have been shown to track esophageal pressure, which is commonly measured to detect changes in inspiratory effort resulting from sleep apnea events (Argod, J., et al., Differentiating obstructive and central sleep respiratory events through pulse transit time. Am J Respir Crit Care Med, vol. 158, 1778-1783, 1998). Argod et al. also demonstrate that PTT signals corresponding to events of sleep apnea vary according to the type of sleep apnea, and may be analyzed in order to classify the apnea event as being either central or obstructive. PTT signals indicative of each type of apnea event will be described in greater detail below, in conjunction with FIGS. 4A-B.

If external processor 110 is employed in conjunction with one of external sensors 116, 118, the ventricular depolarization signal may be transmitted wirelessly, as indicated by the double-headed arrow in FIG. 1, from IMD 100, for example, via a communications module including a telemetry circuit 330 and an antenna 332 (FIG. 2), to a similar communications module of external processor 110. External signal processor 110, in conjunction with sensor 118, may be similar to a pulse-oximetry monitor programmed to calculate PTT, for example, the Datex Cardiocap II; and signal processor 110 may be adapted to also function as an IMD programmer, for example, similar to the Medtronic CareLink® Programmer. Telemetry circuit 330 and antenna 332 of IMD 100 may also function to wirelessly receive the peripheral pulse signals from external signal processor 110 or any of sensors 116, 118, 120 so that microprocessor 224 of IMD 100 may carry out the monitoring method.

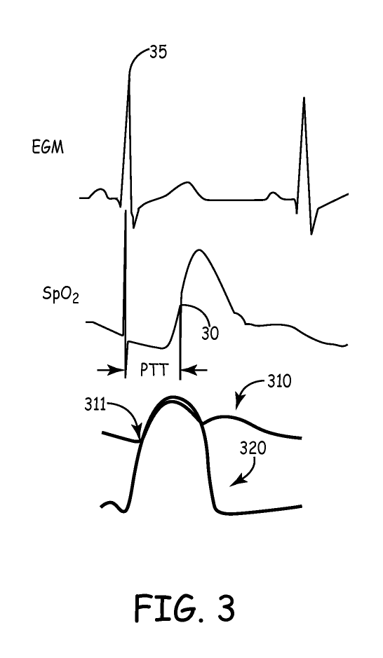

FIG. 3 is a group of tracings illustrating a measure of a single PTT, according to some embodiments of the present invention. FIG. 3 illustrates an EGM trace aligned in time with an oxygen saturation (SpO₂) trace, for example, as recorded via pulse-oximetry; the start of PTT is triggered by a detection of ventricular depolarization, marked at a peak 35 of an R-wave, and an end of PTT is defined by an increase in detected oxygen saturation, marked at a point 30. FIG. 3 further illustrates an aortic pressure trace 310 and an LV pressure trace 320, both traces also being aligned in time with the EGM and SpO₂ traces. Although ventricular depolarization is detected just prior to a point 311 when the aortic valve opens, inclusion of pre-ejection time in PTT has been shown to have no significant impact on the effectiveness of the monitoring method.

Oxygen saturation serves as one type of peripheral pulse signal, for example, being measured by pulse-oximeter sensor 118 clipped to a finger of the patient, or being measured by implanted pulse-oximeter sensor 107 disposed adjacent to subcutaneous pocket arterioles (FIG. 1). Typically, point 30 is either 25% or 50% of a maximum saturation value and is indicative of passage of the arterial pressure pulse. According to alternate embodiments of the present invention, peripheral pulse pressure is measured directly, for example, via one of pressure cuff sensors 116, 120, in order to detect passage of the arterial pressure pulse as the end of PTT.

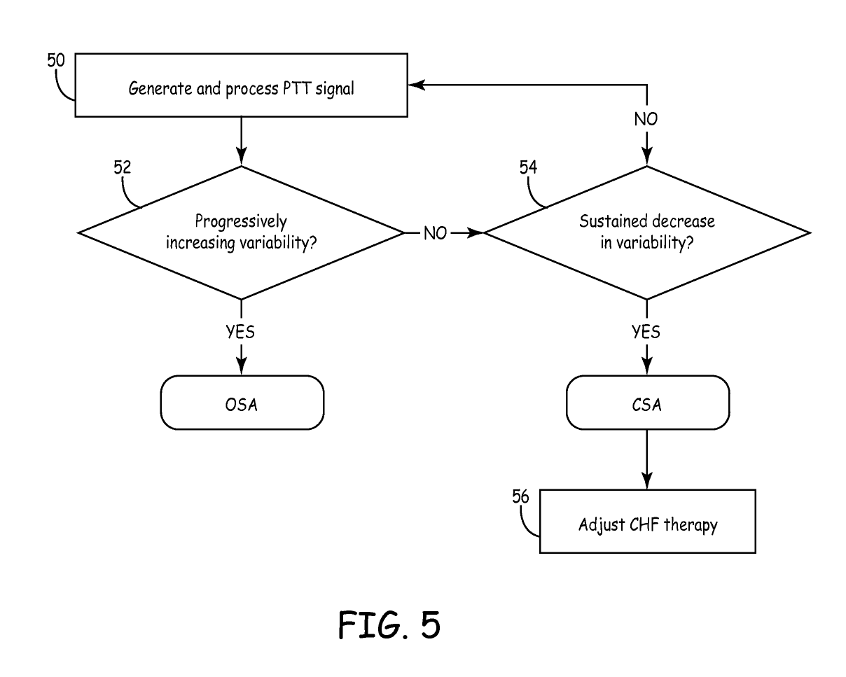

FIGS. 4A-B are plots representative of a PTT signal corresponding to a central sleep apnea (CSA) event, and representative of a PTT signal corresponding to an obstructive sleep apnea (OSA) event, respectively. FIG. 4A illustrates hyperpneic episodes 40 each followed by hypopneic/apneic episodes 42 in which there are sustained decreases in a variability of PTT's, which are typical of CSA events. FIG. 4B illustrates periods of relatively normal respiration 43 each followed by crescendo episodes 45 of progressively increasing variability in PTT's, which are typical of obstructive sleep apnea. According to embodiments of the present invention, PTT signals, such as those shown in FIGS. 4A-B, may be generated using ventricular depolarization signals collected from electrode 114 and peripheral pulse signals collected from any of sensors 107, 116, 118, 120 (FIG. 1), and analyzed via signal processing, which takes place either in microprocessor 224 of IMD 100, or in external signal processor 110, according to pre-programmed methods of the present invention, for example, as outlined by the flow chart in FIG. 5.

FIG. 5 outlines some methods of the present invention in which PTT signals are generated and analyzed to classify apnea events as either OSA or CSA. The detection of CSA in patients receiving CRT, for example, from IMD 100, may be an indicator of worsening CHF that warrants an adjustment of therapy or an administration of additional therapy, for example, as illustrated by a step 56 in FIG. 5. According to some embodiments of the present invention, CSA detection signals are processed by microprocessor 224 in order to trigger adjustments to CRT, via pacing timing and control circuitry 212 (FIG. 2); CRT may be adjusted by changing at least one pacing parameter, for example, a rate and/or interval, of pacing, which may be delivered from electrodes 112 and 114 (FIG. 1), according to methods known to those skilled in the art.

FIG. 5 illustrates an initial step 50 in which a series of consecutive PTT's are measured, for example, over 10 pulse cycles, to generate a PTT signal. According to an embodiment of the present invention, in order to generate the PTT signal, each PTT signal is identified by the detection of a ventricular polarization, which corresponds to the start of the PTT signal, and an increase in detected oxygen saturation, which corresponds to the end of the PTT signal, as described above in reference to FIG. 3, for example.

Step 50 further includes processing of the PTT signal, which is composed of the series of PTT's plotted versus time, in order to evaluate PTT variability over time. According to some embodiments of the present invention, each successive PTT is compared with a preceding PTT in order to determine if there is progressive increase in variability of PTT's within the signal, for example, as illustrated by episodes 45 in FIG. 4B, or if there is a sustained decrease in variability of PTT's within the signal, for example as illustrated by episodes 42 in FIG. 4A. According to an embodiment of the present invention, a sustained decrease in variability of PTT's in the signal is identified when there are sustained decreases in PTT over five or more pulse cycles. Thereforeif such a sustained decrease in variability is detected, absent the detection of progressively increasing variability, a CSA event may be classified. The signal processing of step 50 may employ a Fourier transform function, to calculate an energy of the PTT signal, and then compare the AC signal energy to preset energy thresholds; a signal energy exceeding a preset upper energy threshold may be indicative of progressively increasing PTT variability, while a signal energy below a preset lower energy threshold may be indicative of a sustained decrease in PTT variability absent any episodes of progressively increasing PTT variability. According to the method outlined in FIG. 5, a decision point 52 following signal processing in step 50 either leads to a classification of the apnea event as OSA, if progressively increasing variability in the PTT signal is detected, or leads to a second decision point 54, if progressively increasing variability is not detected. At decision point 54, if a sustained decrease in variability of the PTT signal is detected, decision point 54 leads to a classification of CSA and a subsequent adjustment of CHF therapy, per step 56, for example, via adjustment of at least one pacing parameter; if a sustained decrease in variability is not detected, decision point 54 leads back to step 50 wherein a new series of PTT's are measured and collected into a signal for processing.

According to some embodiments of the present invention, methods outlined by the flow chart of FIG. 5 are triggered by detection of an apnea event, for example, via respiration monitoring wherein a disappearance or reduction in respiratory oscillations is detected. According to an exemplary embodiment, electrode 114 and device housing 105, which acts as a reference electrode, are employed to measure thoracic impedance from which minute volumes may be derived to detect apnea according to cyclical changes in the minute volume. With reference back to FIG. 2, a terminal 305 for housing 105 and terminal 314 for electrode 114 are shown connected to an impedance measurement circuit 215. Circuit 215, being directed by microprocessor 224, applies a series of current pulses between housing 105 and electrode 114 and receives back, for input into microprocessor 224, corresponding potentials, indicative of thoracic impedance, between housing 105 and electrode 114. Aforementioned commonly assigned patent application Ser. No. 10/419,404 describes a method for monitoring minute volume via impedance measurements, as well as alternative methods for monitoring respiration, such as via heart rate sensing. Once an apnea event is detected via the impedance measurements, ventricular depolarization signals are transmitted to one of microprocessor 224 of IMD 100 and external signal processor 110 for the commencement of PTT measurements, per step 50 of FIG. 5. Those skilled in the art will appreciate that embodiments of the present invention can alternatively employ other methods for respiration monitoring to trigger step 50; examples of other methods for respiration monitoring include, without limitation, those that utilize measures, direct or indirect, of airflow, lung volume, and/or pleural pressure.

In the foregoing detailed description, the invention has been described with reference to specific embodiments. However, it may be appreciated that various modifications and changes can be made without departing from the scope of the invention as set forth in the appended claims.

1. A method for monitoring a patient, the method comprising:
measuring a series of consecutive pulse transit times of the patient;
detecting a presence or absence of central sleep apnea according to the measured pulse transit times; and
determining an effectiveness of congestive heart failure therapy based on the detected presence or absence of central sleep apnea, the therapy being provided by electrical stimulation of the patient's myocardial tissue, the stimulation being delivered from a medical device implanted in the patient.

2. The method of claim 1, wherein the measuring comprises:
detecting cardiac ventricular depolarization signals of the patient via an electrode of the implanted medical device;
detecting peripheral arterial pressure pulses of the patient; and
determining a time between each detected ventricular depolarization signal and each subsequent peripheral arterial pressure pulse.

3. The method of claim 2, wherein the peripheral arterial pressure pulses are detected by an external pressure cuff coupled to an arm of the patient.

4. The method of claim 2, wherein the peripheral arterial pressure pulses are detected by an implanted pressure cuff coupled to an artery of the patient.

5. The method of claim 1, wherein the measuring comprises:
detecting cardiac ventricular depolarization signals of the patient via an electrode of the implanted medical device;
detecting peripheral arterial oxygen saturation increases of the patient; and
determining a time between each detected ventricular depolarization signal and each subsequent oxygen saturation increase.

6. The method of claim 5, wherein the peripheral arterial oxygen saturation increase is measured by an external pulse-oximeter sensor coupled to an extremity of the patient.

7. The method of claim 1, wherein detecting the presence of central sleep apnea is based on a detected decrease in variability of pulse transit times sustained over at least five pulse cycles, which is not immediately preceded by a detected progressive increase in variability of pulse transit times.

8. The method of claim 1, further comprising detecting sleep apnea, via respiration monitoring of the patient, prior to measuring the pulse transit times, wherein the detection of sleep apnea triggers the measuring of pulse transit times.

9. The method of claim 8, wherein detecting the presence of central sleep apnea is based on an absence of a detected progressive increase in variability of pulse transit times.

10. The method of claim 8, wherein respiration monitoring comprises measuring thoracic impedance of the patient.

11. A system for monitoring a patient, the system comprising:
an implantable medical device (IMD) for providing electrical stimulation of the patient's myocardial tissue, the IMD including an electrode, a signal processor coupled to the electrode, and a wireless communications module coupled to the signal processor for transmitting the patient's cardiac ventricular depolarization signals detected by the electrode;
an external pulse-oximeter sensor for attachment to an extremity of the patient to measure peripheral arterial oxygen saturation of the patient; and
an external signal processor coupled to the pulse-oximeter sensor and including a wireless communications module for receiving the transmitted depolarization signals from the IMD;
wherein the external signal processor is adapted to:
measure a series of consecutive pulse transit times, each pulse transit time being a time between each depolarization signal and a subsequent rise in oxygen saturation detected by the pulse-oximeter sensor;
detect a presence or absence of central sleep apnea according to the measured pulse transit times; and
determine an effectiveness of congestive heart failure therapy based on the detected presence or absence of central sleep apnea, the therapy being provided by the electrical stimulation of the patient's myocardial tissue.

12. The system of claim 11, wherein the external signal processor detects the presence of central sleep apnea based on a detected decrease in variability of pulse transit times sustained over at least five pulse cycles, which is not immediately preceded by a detected progressive increase in variability of pulse transit times.

13. The system of claim 11, further comprising:
a respiration monitoring device for detecting sleep apnea in the patient, the respiration monitoring device adapted for communication with the communications module of the IMD to trigger transmission of the depolarization signals based on the detection of sleep apnea; and
wherein the external signal processor detects the presence of central sleep apnea based on an absence of detected progressive lengthening of pulse transit times.

14. The system of claim 13, wherein the respiration monitoring device comprises at least two electrodes of the IMD for measuring thoracic impedance of the patient.

15. A system for monitoring a patient, the system comprising:
an implantable medical device (IMD) for providing electrical stimulation of the patient's myocardial tissue, the IMD including an electrode and a signal processor adapted to receive the patient's cardiac ventricular depolarization signals from the electrode and to receive the patient's peripheral arterial pulse signals, the signal processor including pre-programmed instructions for a monitoring method, the monitoring method comprising:
measuring a series of consecutive pulse transit times, each pulse transit time being a time between a depolarization signal of the patient's cardiac ventricular depolarization signals and an immediately subsequent pulse signal of the patient's peripheral arterial pulse signals;
detecting a presence or absence of central sleep apnea according to the measured pulse transit times; and
determining an effectiveness of congestive heart failure therapy based on the detected presence or absence of central sleep apnea, the therapy being provided by the electrical stimulation of the patient's myocardial tissue.

16. The system of claim 15, further comprising a pulse-oximeter sensor adapted to provide the peripheral arterial pulse signals.

17. The system of claim 15, further comprising a pressure sensor adapted to provide the peripheral arterial pulse signals.

18. The system of claim 15, wherein detecting the presence of central sleep apnea is based on a detected decrease in variability of pulse transit times sustained over at least five pulse cycles, which is not immediately preceded by a detected progressive increase in variability of pulse transit times.

19. The system of claim 15, further comprising a respiration monitoring device adapted to detect sleep apnea in the patient and to trigger the monitoring method upon the detection of sleep apnea.

20. The system of claim 19, wherein detecting the presence of central sleep apnea is based on an absence of a detected progressive increase in variability of pulse transit times.

21. A method for providing cardiac resynchronization therapy to a patient, the therapy delivered via pacing from an implanted medical device, the method comprising:
measuring a series of consecutive pulse transit times of the patient;
detecting a presence or absence of central sleep apnea according to the measured pulse transit times; and
adjusting at least one pacing parameter of the implanted medical device, if the presence of central sleep apnea is detected.

22. The method of claim 21, wherein the measuring comprises:
detecting the patient's cardiac ventricular depolarization signals via an electrode of the implanted medical device;
detecting the patient's peripheral arterial pressure pulses; and
determining a time between each detected ventricular depolarization signal and each subsequent peripheral arterial pressure pulse.

23. The method of claim 21, wherein the measuring comprises:
detecting cardiac ventricular depolarization signals of the patient via an electrode of the implanted medical device;
detecting peripheral arterial oxygen saturation increases of the patient; and
determining a time between each detected ventricular depolarization signal and each subsequent oxygen saturation increase.

24. The method of claim 21, wherein detecting the presence of central sleep apnea is based on a detected decrease in variability of pulse transit times sustained over at least five pulse cycles, which is not immediately preceded by a detected progressive increase in variability of pulse transit times.

25. The method of claim 21, further comprising detecting sleep apnea, via respiration monitoring of the patient, prior to measuring the pulse transit times, wherein the detection of sleep apnea triggers the measuring of pulse transit times.

26. The method of claim 25, wherein detecting the presence of central sleep apnea is based on an absence of a detected progressive increase in variability of pulse transit times.

27. The method of claim 25, wherein respiration monitoring comprises measuring thoracic impedance of the patient.
